(19) United States
(12) Patent Application Publication
(10) Pub. No.: US 2022/0096275 A1
(43) Pub. Date: Mar. 31, 2022

(54) PERSONAL PROTECTIVE EQUIPMENT

(71) Applicant: Derek C. Jackson, Columbus, OH (US)

(72) Inventor: Derek C. Jackson, Columbus, OH (US)

(21) Appl. No.: 17/489,549

(22) Filed: Sep. 29, 2021

Related U.S. Application Data
(60) Provisional application No. 63/084,614, filed on Sep. 29, 2020.

(51) Int. Cl. A61F 9/02; A62B 23/02; A62B 18/08; A42B 5/00

(52) U.S. Cl. CPC A61F 9/029; A42B 5/00; A62B 18/084; A62B 23/02

(57) ABSTRACT

One or more embodiments of a device including a head piece, a head covering, a visor, and a veil are disclosed. The head piece is sized and arranged to sit on a head of a wearer when the device is worn on the head of the wearer. The head covering is connected to the head piece and is connected over the head piece and is configured to rotate relative to the head piece. The visor is connected to the head covering, the visor being at least partially transparent. The veil connects to the visor, the veil including a protective material.

### CROSS REFERENCE TO RELATED APPLICATIONS

This application claims priority to provisional application No. 63/084,614, which was filed on Sep. 29, 2020, which is incorporated in its entirety.

### FIELD OF THE DISCLOSURE

The present invention relates to devices for personal protection, specifically personal protection from airborne contaminants.

### BACKGROUND

Various forms of head and facial protection exist to protect a human wearer. Some forms of head protection such as beekeeper suits and mosquito nets cover the head and protect the head from insects and sometimes sunlight. Further these types of head protection can be uncomfortable for the wearer when the head is rotated because the head covering cannot effectively rotate independently of the head of the user and the netting or other material of head protection twist and resist the rotation. Thus, the wearer feels an uncomfortable restriction from rotating the head. Also, these forms of protection provide very little if any protection from airborne contaminants.

Some forms of facial protection such as surgical masks provide some protection from airborne contaminants but are very uncomfortable for the wearer who must work harder to breathe through the mask. Also, moisture and heat uncomfortably build up in and on the mask. Further, air is often pulled into the mouth of the wearer around the mask which negates some of the protective qualities of the mask.

### SUMMARY

One or more embodiments are provided below for a device for protecting a wearer from airborne contaminants. The device may include a head piece, a head covering, a visor and a veil. The head piece may be sized and arranged to sit on a head of a wearer when the device is worn on the head of the wearer. The head covering may be connected to the head piece and may be connected over the head piece and may be configured to rotate relative to the head piece. The visor may be connected to the head covering. The visor may be at least partially transparent. The veil may be connected to the visor. The veil may include a protective material.

The device may provide significant advantages over the devices known in the art. The device may provide superior protection for the head of the wearer by completely covering the head and neck of the wearer while filtering air through a filter. The device may also provide superior comfort because the device allows the wearer to rotate his/her head independently of portions of the personal protection device.

Other advantageous features as well as other aspects and advantages of the invention will be apparent from the following description and the appended claims.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure.

FIG. 1A shows an example front view of a Personal Protective Equipment (PPE).

FIG. 1B shows an example side view of the PPE.

FIG. 1C shows an example top view of the PPE.

FIG. 2 shows an example of an exploded front view of the head covering and head gripper of the PPE.

FIG. 3 shows an example bottom view of the head gripper.

FIG. 4A and FIG. 4B show an example cross section view of the connection between the visor and veil.

FIG. 5 shows an example front view of the PPE on a wearer.

FIG. 6 shows an example front-top view of a second embodiment of the PPE.

FIG. 7 shows an exploded view of the second embodiment of the PPE.

### DETAILED DESCRIPTION

In the Summary above and in this Detailed Description, the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, among others, are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm and upper limit is 100 mm.

Certain terminology and derivations thereof may be used in the following description for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “coupled to” as used herein may mean a direct or indirect connection via one or more components.

Referring now to the drawings and the following written description of the present invention, it will be readily understood by those persons skilled in the art that the present invention is susceptible to broad utility and application. Many embodiments and adaptations of the present invention other than those herein described, as well as many variations, modifications, and equivalent arrangements will be apparent from or reasonably suggested by the present invention and the detailed description thereof, without departing from the substance or scope of the present invention. This disclosure is only illustrative and exemplary of the present invention and is made merely for purposes of providing a full and enabling disclosure of the invention.

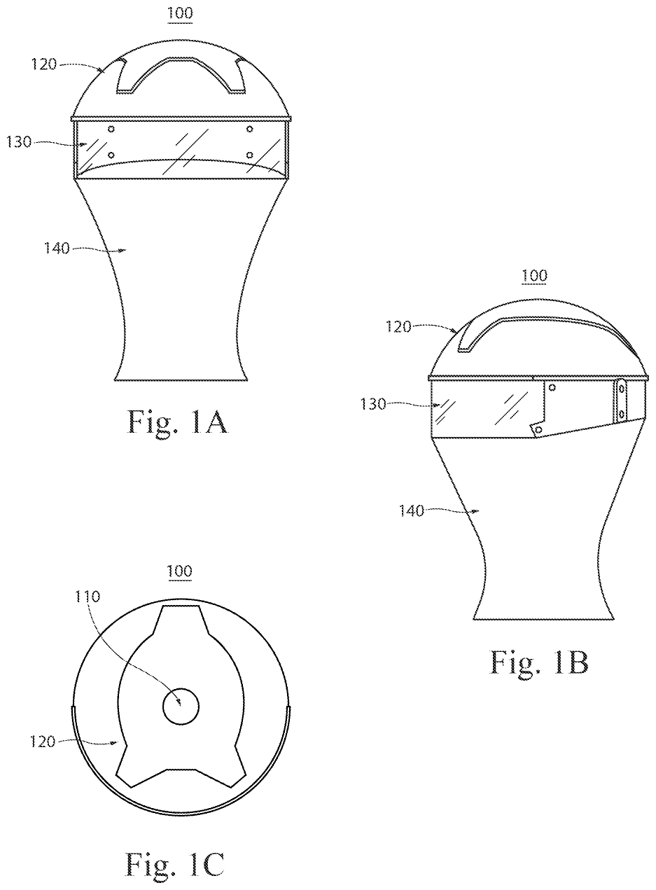

FIG. 1A shows an example front view of a Personal Protective Equipment (PPE) 100. FIG. 1B shows an example side view of the PPE. FIG. 1C shows an example top view of the PPE. The PPE 100 may include a head covering 120, a visor 130, a veil 140, and a head gripper 150 (not shown in these figures). As described in further detail below, the head gripper 150 is connected to the head covering 120 by a connector 110. The connector 110 may be on top of the head covering 120. The head covering 120 may be made of a stiff material such as plastic, felt, vinyl, or other material suitable for covering a head and supporting the weight of a visor 130 and veil 140. The connector 110 may be made of a similar material as the head protector 120 that is also suitable for connecting the head gripper 150 to the head protector 120. The head covering 120 may be configured to cover and protect a top (crown) of a head of a wearer by sitting over the head of the wearer when worn. The head cover 120 may extend out from the head of the wearer to provide a comfortable distance from the face of the wearer to the visor 130 (this also helps prevent fogging on the visor 130).

The visor 130 may be made of a transparent or semi-transparent material such as transparent plastic or glass. The visor 130 may be tinted, include a tinted layer, or embedded with UV protection to provide protection from ultraviolet light or bright light. The visor 130 may be permanently attached to the head covering 120, for example by gluing, sewing, or other attachment mechanisms. The visor 130 may form a 360-degree ring around the head of a wearer of the PPE 100, allowing the wearer to see out of the PPE 100 independently of a direction the head of the wearer is facing relative to the PPE 100. In other embodiments the visor may extend 180 degrees or more around the head of the wearer. And in still other embodiments, the visor 130 may extend 90 degrees or more around the head of the wearer. The visor 130 may extend down about 3-6 inches from the head cover 120. The visor 130 may be arranged and sized such that the wearer can see out of the visor directly in front of the eyes of the wearer.

As will be described in further detail below, the visor 130 may be removably attached to the veil 140. The veil 140 may include a protective material. The protective material may be a filtering material that allows air to pass through and also acts to filter out contaminants (e.g., water particles containing viruses and/or bacteria, allergens, and air pollutants) from the air passing through the veil 140. For example, the veil 140 may include a HEPA (high efficiency particulate air) filter material or other similar material. In another embodiment, the protective material may be netting for keeping insects such as mosquitoes and bees out. The head covering 120 and the visor 130 may not allow substantial air flow and the veil 140 may filter substantially all air passing into and out of the PPE 100. The veil 140 may extend down about 6-18 inches from the visor 130. The veil 140 may be configured to have a portion of the veil 140 rest on the collar of the wearer, when the wearer wears the PPE 100. The veil 140 may have a reduction in diameter and then flare out such that the wearer may comfortably slip the veil under the collar of a shirt with a portion of the veil being under the shirt of the wearer resting on the collar and back of the wearer. The veil 140 may be sized and arranged to completely encircle a portion of the head of the wearer (about from the nose down) and the neck of the wearer.

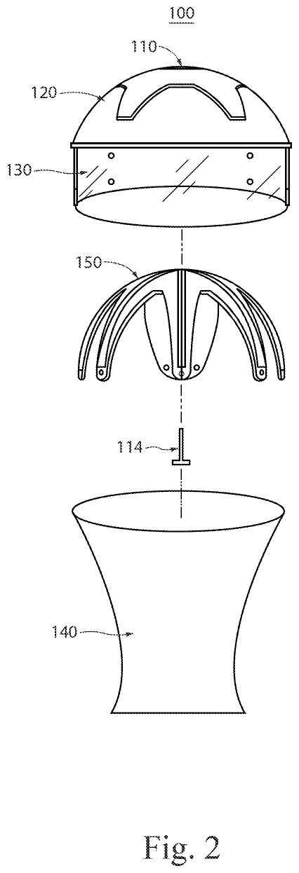

FIG. 2 shows an example of an exploded front view of the head covering 120 and head gripper 150 of the PPE 100. The PPE 100 may include the head gripper 150 which is attached to the head cover 120 via the axle 114 attached to the connector 110. The head covering may include a hole at a top of the head cover 120 where the axle 114 passes through to connect to the connector 110. The hole may have a grommet reinforcing the hole to reduce tearing or stretching of the material of the head covering 120. The axle 114 may be a rivet or other similar device that allows for one or both of the head gripper 150 and the head covering 120 to rotate independently from the axle 114. In this way, the axle 114 allows the head gripper 150 to rotate independently of the head covering 120. Thus, via the axle 114, the head gripper 150 and the head cover 120 may be rotatably attached. The axle 114 may be about one quarter to one inch high with a diameter of about one quarter to one inch. The axle 114 and connector 110 may be any one of various connecting devices which allow the head gripper 150 to rotate independently of the head covering 120 (e.g., clevis pin, screw and nut, bolt and friction held cap, rivet, etc.).

The head gripper 150 may have a general bowl shape with portions missing such that fingers extend downward. The general bowl shape of the head gripper 150 may have a diameter less than the diameter of the head cover 120 such that the head gripper 150 may sit in the head cover 120. The head gripper 150 may be sized and arranged to grip onto a head of a wearer when worn on the head of the wearer such that the head gripper 150 rotates less than the head cover 120 when the wearer rotates his/her head. The difference in rotation being enabled by the connection of the head cover 120 to the head gripper 150 by the axel 114.

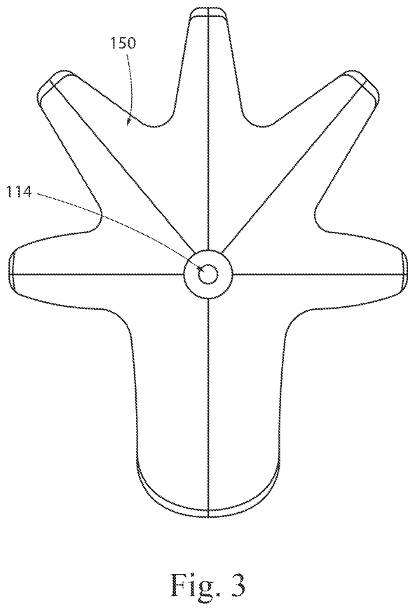

FIG. 3 shows an example bottom view of the head gripper 150. A base of the axel 114 is also shown. The axel passes through the head griper 150 so it may attach to the head cover 120. The head gripper 150 may have a general inverse bowl shape that is configured to fit snugly on the head of the wearer such that the head gripper 150 moves with the movements of the head of the wearer. The first head gripper 150 may be made of a stiff material such as plastic, felt, fabric with a reinforcing frame, etc. The reinforcements may be metal, plastic, or other stiff material extensions along a part of or the entire length of the fingers of the head gripper 150. The stiffness of the material may help to dissipate the weight of the PPE 100 over a larger area of the head of the wearer to prevent discomfort from too much pressure on one point on the head. The head gripper 150 may include ventilation holes (not shown) and padding (not shown) to increase the comfort of the wearer. The gaps between the fingers of the head gripper 150 may also act as ventilation areas for greater comfort.

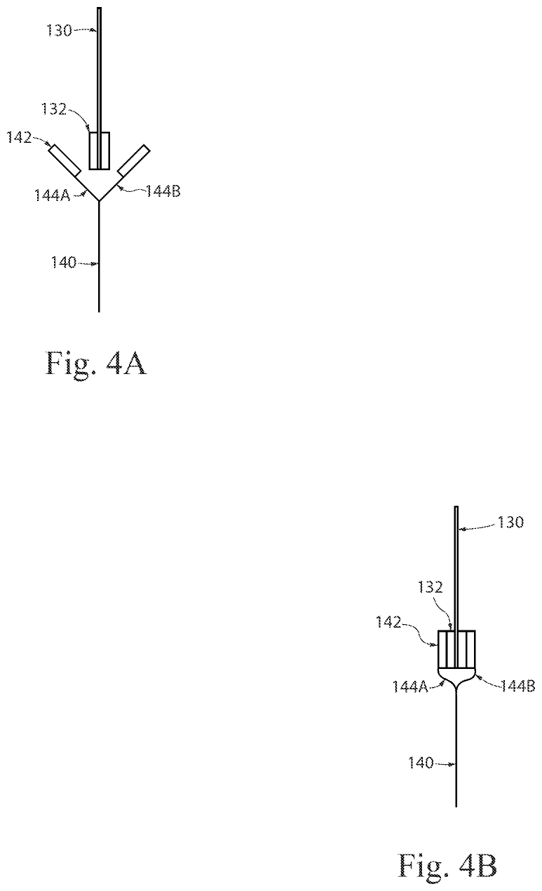

FIG. 4A and FIG. 4B show an example cross section view of the connection between the visor 130 and veil 140. FIG. 4A shows the disconnected visor 130 and veil 140. The visor 140 may include an attachment 132 on each side of the visor 130 at a bottom of the visor 130. The attachments 132 may be either the hook or loop component of a hook and loop attachment or other similar attachment devices. The veil may include a first connection extension 144A and a second connection extension 144B. Each of the first connection extension 144A and the second connection extension 144B may extend from the top of the veil 140 and include an attachment 142. The attachments 142 may be the complimentary hook or loop of the hook and loop attachment to the attachments 132. Having two connections between the veil 140 and the visor 130 helps to prevent inadvertent decoupling of the veil 140 from the visor 130.

FIG. 4B shows the connected visor 130 and veil 140. As shown, the visor 130 and veil 140 may be connected via the attachments 132, 142 both on an interior of the visor 130 and an exterior of the visor 130. The detachability of the veil 140 is advantageous for cleaning or replacing the veil. Other connections such as rubber splines, buttons, snaps, etc. may also be used for attaching the visor 130 to the veil 140. If the visor 130 does not extend completely around the head of the wearer, the veil 140 may also be connected to portions of the head cover 120 using the same or similar means of connection as the visor 130.

The PPE 100 may be worn on and around the head of a wearer to protect the wearer from airborne contaminants, sunlight, wind, cold weather, etc. The PPE 100 provides improved comfort for the wearer by allowing the wearer to rotate the head gripper 150 independently of the head covering 120, providing protection at a comfortable distance from the face of the wearer, minimizing pressure on a single location on the head of the wearer, etc.

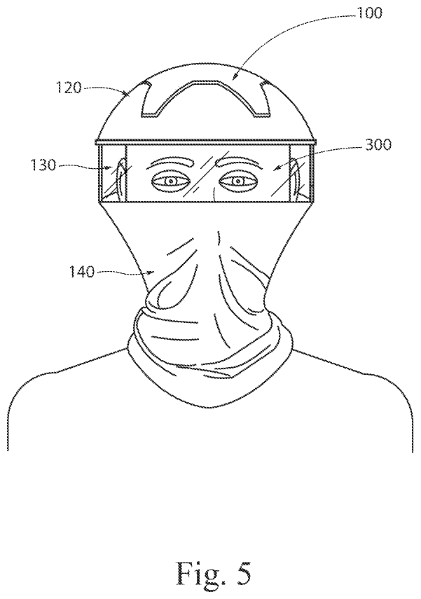

FIG. 5 shows an example front view of the PPE on a wearer 300. The head covering 120 may cover the crown of the wearer's 300 head and the eyes of the wearer 300 may be able to see out of the visor 120 straight ahead. The veil 140 may completely surround the lower part of the head of the wearer 300 and the neck of the wearer 300. The head and neck of the wearer may be completely inserted into the PPE 100. The veil 140 may rest on the collar of the wearer 300.

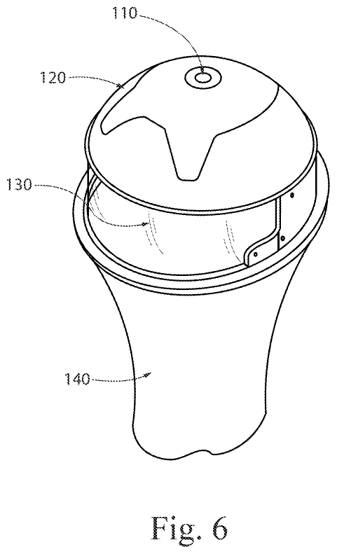

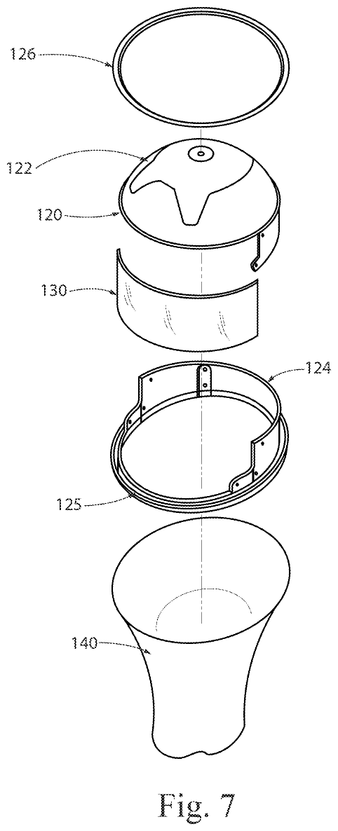

FIG. 6 shows an example front-top view of a second embodiment of the PPE 100. FIG. 7 shows an exploded view of the second embodiment of the PPE 100. The second embodiment may be the same as the embodiment of the FIGS. 1A-C except as described below. In the second embodiment the PPE 100 may have a different manner of attaching the veil 140 to the head cover 120. The head cover 120 may include un upper portion 122, a lower portion 124, and a seal 126. The upper portion 122 may be sized and arranged to cover the crown of the head of the wearer. The lower portion 124 may include a ring that connects under the visor 130 to secure the visor 130 to the upper portion 122. The lower portion 124 may connect to the upper portion 124 at a back of the PPE 100. The visor 130 may be secured between the upper portion 122 and the lower portion 124 of the head cover 120. The lower portion may include a groove 125 that is defined in the ring of the lower portion 124 and extends around the ring of the lower portion 124. To attach the veil 120 to the head covering 140 the upper most portion of the veil 140 may be inserted into the groove 125 and the seal 126 may be placed in the groove over the upper most portion of the veil 140 to fix the veil 140 in the groove 125. The seal 126 may be made of rubber silicone or another material with a high enough friction coefficient to secure the veil 140 in the groove 125.

[symbol]

In one embodiment, a device (e.g., PPE 100) may include a head piece (e.g., head gripper 120), a head cover 120, a visor 130, and a veil 140. The head piece may be sized and arranged to sit on a head of a wearer when the device is worn on the head of the wearer. The head cover 120 may be connected to the head piece, may be connected over the head piece, and may be configured to rotate relative to the head piece. The visor 130 may be connected to the head cover 120, the visor 130 being at least partially transparent. The veil 140 may connect to the visor 130. The veil 140 may include a protective material.

The head cover 120 may be sized and arranged to cover a crown of the head of the wearer. The visor 130 may extend down from the head cover 120 and may be sized and arranged to allow a wearer to see through the visor 130 when the wearer's head is facing forward. The veil 140 may extend downward from the visor 130 and the protective material of the veil 140 may be an air filtering material. The veil 140 may be sized and arranged to filter air breathed by the wearer. The veil 140 may be sized and arranged to encircle the head or the neck of the wearer.

The visor 130 may be sized and arranged to extend at least 90 degrees around the head of the wearer when the device is worn by the wearer. The veil 140 may be removably connected to the visor 130. The veil 140 may be removably connected to the head cover 120 via a groove 125 in the head cover 120 and seal 126 placed into the groove 125 with a portion of the veil 140. The head piece may be sized and arranged to grip the crown of the head of the wearer such that when the head of the wearer is rotated the head piece rotates more than the head cover 120.

The head cover 120 may be sized and arranged to cover a crown of a head of a wearer when the device is worn by the wearer. The head piece may be rotatably connected to the head cover 120 and sized and arranged to grip the crown of the head of the wearer such that when the head of the wearer is rotated the head piece rotates more than the head cover 120. The head piece may be arranged to rotate independently of the head cover 120, the visor 130, and the veil 140. The head piece may be connected to the head cover 120 by an axle 114 configured and arranged to allow the head piece and head cover 120 to rotate independently.

Many different embodiments of the inventive concepts have been shown. A person of ordinary skill in the art will appreciate that the features from different embodiments may be combined or replaced with other features from different embodiments.

Advantageously, the PPE 100 may provide superior protection for the head of the wearer by completely covering the head and neck of the wearer while filtering air through a filter (veil 140). The PPE 100 may also provide superior comfort because the device allows the wearer to rate his/her head with the head gripper 150 independently of the head cover 120, the visor 130, and the veil 140.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention.

The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. A device comprising:
a head piece sized and arranged to sit on a head of a wearer when the device is worn on the head of the wearer;
a head covering connected to the head piece and over the head piece and configured to rotate relative to the head piece;
a visor connected to the head covering, the visor being at least partially transparent; and
a veil connected to the visor, the veil including a protective material.

2. The device of claim 1, wherein the head covering is sized and arranged to cover a crown of the head of the wearer.

3. The device of claim 2, wherein the visor extends down from the head covering and is sized and arranged to allow the wearer to see through the visor when the wearer's head is facing forward.

4. The device of claim 3, wherein the veil extends downward from the visor and the protective material is an air filtering material.

5. The device of claim 4, wherein the veil is sized and arranged to filter air breathed by the wearer.

6. The device of claim 5, wherein the veil is sized and arranged to encircle the head or a neck of the wearer.

7. The device of claim 6, wherein the visor is sized and arranged to extend at least 90 degrees around the head of the wearer when the device is worn by the wearer.

8. The device of claim 7, wherein the veil is removably connected to the visor.

9. The device of claim 8, wherein the veil is removably connected to the head cover via a groove in the head cover and seal placed into the groove with a portion of the veil.

10. The device of claim 1, wherein the head piece is sized and arranged to grip the crown of the head of the wearer such that when the head of the wearer is rotated the head piece rotates more than the head covering.

11. A device comprising:
a head covering sized and arranged to cover a crown of a head of a wearer when the device is worn by the wearer;
a head piece rotatably connected to the head covering and sized and arranged to grip the crown of the head of the wearer such that when the head of the wearer is rotated the head piece rotates more than the head covering;
a visor connected to the head covering, the visor being at least partially transparent; and
a veil connected to the visor, the veil including a protective material.

12. The device of claim 11, wherein the head piece is arranged to rotate independently of the head covering, the visor, and the veil.

13. The device of claim 12, wherein the visor extends down from the head covering and is sized and arranged to allow a wearer to see through the visor when the wearer's head is facing forward.

14. The device of claim 13, wherein the veil extends downward from the visor and the protective material is an air filtering material.

15. The device of claim 14, wherein the veil is sized and arranged to filter air breathed by the wearer.

16. The device of claim 15, wherein the veil is sized and arranged to encircle the head or a neck of the wearer.

17. The device of claim 16, wherein the visor is sized and arranged to extend at least 90 degrees around the head of the wearer when the device is worn by the wearer.

18. The device of claim 17, wherein the veil is removably connected to the visor.

19. The device of claim 18, wherein the veil is removably connected to the head cover via a groove in the head cover and seal placed into the groove with a portion of the veil.

20. The device of claim 11, wherein the head piece is connected to the head cover by an axel configured and arranged to allow the head piece and head cover to rotate independently.
